SEQUENCE LISTING (7 sequences)

SEQ ID NO: 1 - DNA, 64 bp, Artificial
  Description of Artificial Sequence primer
agacgaatcaacaaagaaccgttatacatcgcgtctatacctgtgacggaagatcacttcgcag

SEQ ID NO: 2 - DNA, 63 bp, Artificial
  Description of Artificial Sequence primer
cggagcgcgaaaatatagggagtatgcggtggttgcaattacgccccgccctgccactcatcg

SEQ ID NO: 3 - DNA, 935 bp, Escherichia coli
agacgaatcaacaaagaaccgttatacatcgcgtctatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaattgcaaccaccgcatactccctatattttcgcgctccg

SEQ ID NO: 4 - DNA, 19 bp, Artificial
  Description of Artificial Sequence primer
cagaagtgtctgtaccggt

SEQ ID NO: 5 - DNA, 21 bp, Artificial
  Description of Artificial Sequence primer
aatgtgctgttaatcacatgc

SEQ ID NO: 6 - DNA, 1492 bp, Escherichia coli
cagaagtgtctgtaccggtaataaagaaacgcttcagcatcactaactccaccgttatgcttcacaaatataaaccaggaaaataattaaccttgaaagtctaagttatgctttcctggcccaaattgagatagcgcaaattttggtagaacagttaaaaaatgttaaccctgcaacagacgaatcaacaaagaaccgttatacatcgcgtcttttaccagtgcagcgcctgccatcgtgccctggttagaaaactgagtactctcaacgctgatgtgctgactatacgcaggaagggcctgctgacggatgctgtctgagatgaccgggaagaggatatctgccgctttacttaacggtgagccaatcagtattttttgtgggttaaataaattcaccatgatggcaagaatgcgcccgacatgcgcgcccaccccggtaatgatgtcttttgccagtagatcgccgcgcaatgccgcctgacacaatgagtccacggttaacggttgtccatgtaacatcgagctcatggattgattaagacgcagctgtgccagctcaagaatactgtccacgctggcgatggtttcgaggcagccgtgattcccgcaataacagcgtttcccatacgggtcgacctgtgtgtggcctatttccacgagactactgctgcctgcgtgtagcagatgaccatcggtaatgacgcccgcccccacgttgtgatcgataaccacctgaatcacatcgcgcgccccgcgtgaggcaccaaacaaggcctctgccatcgtccatgcgctgatatcatgctgaatataaaccggaacgccggtatgctgctccagcgcctcgccgagcggcatctcttttacatcctcgtagaacggcatgcgatgtacaataccattttccgtatcaataattcccggcaaggttatggcaatcgaagttagacgctcaagttttttctggtggcggataaaaaactgatcgatatgggaaataatacgatccagcaatggcaagtcatcttttaacgccagttcctgcgactcttccaccaccagtttgctgctcagatcgcgcagagcaaggaaaatctccccgcgactaatgcgcagagaaagatagtgccaggcttcagtttcaaccaccagccccaccgccggacggccacggttccccgcttctttgatttccagctcttgcaccaggtgtgcttcgagcatctcacggacaattttagtgatactggcaggagccagttgcgccagacgggaaagatcgatacgcgagactggaccaagctgatcaatcaggcgataaaccgcgcccgcgttggtctgctttatttgatcaatgtgcccaggctggttttcagcaaccaccgcatactccctatattttcgcgctccgaaataatctgtaggctatggtgaagcacttcaatacgtgtcgtcaaatttttacttaggcatgtgattaacagcacatt

SEQ ID NO: 7 - DNA, 1191 bp, Escherichia coli
cagaagtgtctgtaccggtaataaagaaacgcttcagcatcactaactccaccgttatgcttcacaaatataaaccaggaaaataattaaccttgaaagtctaagttatgctttcctggcccaaattgagatagcgcaaattttggtagaacagttaaaaaatgttaaccctgcaacagacgaatcaacaaagaaccgttatacatcgcgtctatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaattgcaaccaccgcatactccctatattttcgcgctccgaaataatctgtaggctatggtgaagcacttcaatacgtgtcgtcaaatttttacttaggcatgtgattaacagcacatt

(19) United States
(12) Patent Application Publication
(10) Pub. No.: US 2004/0229320 A1
(43) Pub. Date: Nov. 18, 2004

(54) Method for procucing L-amino acid using bacterium, belonging to the genus Escherichia, lacking active mlc gene

(76) Inventors: Natalia Viktorovna Stoynova, Moscow (RU); Elena Viktorovna Sycheva, Moscow (RU); Aleksandra Yurievna Skorokhodova, Moscow (RU); Yuri Ivanovich Kozlov, Moscow (RU)

Correspondence Address: AJINOMOTO CORPORATE SERVICES, LLC, INTELLECTUAL PROPERTY DEPARTMENT, 1120 CONNECTICUT AVE., N.W., WASHINGTON, DC, 20036, US

(21) Appl. No.: 10/654,898

(22) Filed: Sep. 5, 2003

(30) Foreign Application Priority Data
Sep. 6, 2002 (RU) 2002123822

(51) Int. Cl. C12P 13/04; C12N 1/21

(52) U.S. Cl. 435/106; 435/252.33

(57) ABSTRACT

The invention relates to a bacterium comprising an inactive mlc gene which produces an L-amino acid, such as L-threonine,

### BACKGROUND OF THE INVENTION

1. Field of the Invention

The present invention relates to the microbiological industry and novel strains of Escherichia coli and fermentation processes involving these microorganisms. More specifically the present invention relates to genetically-modified Escherichia coli strains and the use thereof for production of amino acids, specifically members of the aspartate family of amino acids, such as threonine. Furthermore, the present invention also relates to a method for producing L-amino acid using bacterium in a glucose-containing medium, belonging to the genus Escherichia, wherein mlc gene is inactivated.

2. Description of the Related Art

Mlc protein is a global regulator (repressor) of carbohydrate metabolism (Decker et al, Mol Microbiol 1998, 27:2:381-90; Kimata et al, Mol Microbiol, 1998, 29:6:1509-19; Plumbridge, Mol Microbiol, 1998, 27:2:369-80). It was shown Mlc protein regulates expression of several genes and operons. There are ptsG gene (Kimata et al, Mol Microbiol, 1998, 29:6:1509-19; Plumbridge, Mol Microbiol, 1998, 29:4:1053-63; Kim et al, J Biol Chem, 1999, 274:36:25398-402; Plumbridge, Mol Microbiol 1999, 33:2:260-73; Tanaka et al, Genes Cells, 1999, 4:7:391-9) encoding membrane-bound subunit, IICB(Glc), of glucose phosphotransferase system (PTS), ptsHIcrr operon encoding general PTS proteins (Kimata et al, Mol Microbiol, 1998, 29:6:1509-19; Plumbridge, Mol Microbiol, 1998, 29:4:1053-63; Kim et al, J Biol Chem, 1999, 274:36:25398-402; Plumbridge, Mol Microbiol 1999, 33:2:260-73; Tanaka et al, Genes Cells, 1999, 4:7:391-9), manXYZ operon encoding enzyme II of mannose PTS (Plumbridge, Mol Microbiol, 1998, 29:4:1053-63), malT gene encoding the activator of maltose regulon (Decker et al, Mol Microbiol 1998, 27:2:381-90).

Genes of mlc regulon are also positively regulated by CRP-cAMP complex (Chapon and Colb, J. Bacteriol., 1983, 156:1135-43; Decker et al, Mol. Microbiol., 1998, 27:2:381-90; Kimata et al, Mol. Microbiol., 1998, 29:6:1509-19; Plumbridge, Mol. Microbiol., 1998, 27:2:369-80; Plumbridge, Mol. Microbiol., 1998, 29:4:1053-63; Kim et al, J. Biol. Chem, 1999, 274:36:25398-402; Plumbridge, Mol. Microbiol 1999, 33:2:260-73; Tanaka et al, Genes Cells, 1999, 4:7:391-9).

Regulation of mlc gene transcription is remarkably complicated. First, it is negatively regulated by Mlc protein itself. Unphosphorylated EIICB (Glc) (product of ptsG gene) can sequester Mlc protein from its binding site by direct protein-protein interaction and therefore induce expression of mlc regulon in response of glucose (Tanaka et al, EMBO J, 2000, 19:20, 5344-52; Lee et al, EMBO J, 2000, 19:20:5353-61; Nam et al, EMBO J, 2001, 20:3:491-8). Second, transcription of mlc gene is performed by two promoters P1 and P2 (Shin et al, J. Biol. Chem. 2001, 276:28:25871-75). Promoter P1 is recognized only by RNA polymerase, containing the housekeeping sigma factor σ⁷⁰(Eσ⁷⁰), while the promoter P2 can be recognized by both Eσ⁷⁰ and Eσ³² containing the heat shock sigma factor. Thus mlc gene belongs to a class of genes, transcribed from the multiple promoters including one recognized by RNA polymerase associated with the alternative sigma factor in order to respond to various environmental conditions. In addition, a highly conserved CRP-binding site present within the mlc promoter (Shin et al, J. Biol. Chem. 2001, 276:28:25871-75).

In view of the discussion above, there remains a need in the art for efficient and increased production of amino acids such as threonine. In the populations of Escherichia coli growing in the glucose-limited environment, the polygenic mutations in mgl, mlc and malT genes were found (Manch K., Genetics, 1999, 153:1:5-12). However, there have been no reports or suggestions of inactivation of the mlc gene in bacterium grown in a glucose-containing medium for the purpose of increasing amino acid production.

### SUMMARY OF THE INVENTION

It is therefore an object of present invention to enhance the productivity of L-amino acid producing strains and to provide a method for producing L-amino acid using these strains.

The inventors of the present invention considered that the transport of carbohydrates provided by PTS may be the rate limiting step in overproduction of some amino acids and that the inactivation of the product of mlc gene, which negatively regulates PTS gene expression, seems to be necessary for increasing amino acid production. Based on such concept, the inventors assiduously studied and found that the inactivation of mlc gene encoding the repressor of carbohydrate metabolism can enhance production of L-amino acid, such as L-threonine in a bacterium belonging to the genus Escherichia.

Other objects, features, and advantages of the present invention will be set forth in the detailed description of the preferred embodiments that follows, and in part will be apparent from the description or may be learned by practice of the invention. These objects and advantages of the invention will be realized and attained by the methods particularly pointed out in the written description and claims herein.

It is an object of the present inventions to provide an L-amino acid-producing bacterium belonging to the genus Escherichia, wherein the bacterium is grown in a glucose-containing medium and has been modified to have a mlc gene inactivated.

It is a further object of the invention to provide an L-amino acid producing bacterium belonging to the genus Escherichia, wherein the bacterium has been modified to have a mlc gene inactivated, and wherein L-amino acid is L-threonine.

It is still a further object of the invention to provide the L-amino acid producing bacterium belonging to the genus Escherichia, wherein the bacterium has been modified to have a mlc gene inactivated, and wherein L-amino acid is L-threonine, and wherein the bacterium has been modified to have enhanced expression of L-threonine operon.

It is yet another object of the invention to provide a method for producing L-amino acids, which method comprises the steps of:

cultivating an L-amino acid-producing bacterium belonging to the genus Escherichia in a glucose-containing medium, wherein the bacterium has been modified to have a mlc gene inactivated to produce and accumulate L-amino acid in the medium, and

collecting said L-amino acid from the medium.

It is another object of the invention to provide a method as stated above, wherein L-amino acid is L-threonine.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF DRAWINGS

FIG. 1 shows the relative position of the primers mlcIL and mlcIR on plasmid pACYC184 used for amplification of cat gene.

FIG. 2 shows the construction of chromosomal DNA fragment comprising inactivated mlc gene.

### DESCRIPTION OF THE PREFERRED EMBODIMENTS

In a first embodiment, the present is directed to novel bacterial strains which may be used in fermentation processes for the production of amino acids.

The bacterium of the present invention is an L-amino acid producing bacterium belonging to the genus Escherichia, wherein the bacterium has been modified to have mlc gene inactivated.

In the present invention, “L-amino acid producing bacterium” means a bacterium which has an ability to accumulate L-amino acid in a medium when the bacterium of the present invention is cultured in the medium. The L-amino acid producing ability may be imparted or enhanced by breeding. The term “L-amino acid producing bacterium” used herein also means a bacterium, which is able to produce and accumulate L-amino acid in a culture medium in an amount larger than a wild type or a parental strain of E. coli, such as E. coli K-12 strain.

The term “a bacterium belonging to the genus Escherichia” means that the bacterium is classified as the genus Escherichia according to the classification known to a person skilled in the microbiology. Preferably, the bacterium used in the present invention are strains of Escherichia coli (E. coli). More preferably, the strains used in the present invention are chosen from, for example, the E. coli strains described by Neidhardt, F. C. et al. (Escherichia coli and Salmonella typhimurium, American Society for Microbiology, Washington D.C., 1208, Table 1). A particularly preferred example of the inventive strains is any L-threonine producing strain of E. coli.

The term “mlc gene is inactivated” or “inactive mlc gene” means that mlc gene is modified in such a way that the modified gene encodes a mutant protein with decreased activity, or in the alternative, a completely inactive protein. Another possibility is that the modified DNA region is unable to provide the natural expression of Mlc protein due to deletion of a part of the gene or modification of adjacent region of the gene.

Inactivation of mlc gene coding for the repressor of PTS brings an increase in the supply of carbon source, such as glucose, into the cell of L-amino acid producing bacterium.

mlc gene codes for Mlc protein, which is a global regulator of carbohydrate metabolism. Nucleotide sequence of mlc gene from various organisms has been reported. Among them, for example, mlc gene from E. coli can be used in the present invention. The E. coli mlc gene (gi:16129552; numbers 1665368 to 1666588 in the GenBank accession number NC_{—}000913.1) is located between b 1593 and ynfL genes on the chromosome of E. coli strain K-12 and codes for E. coli Mlc protein (406 amino acid residues).

The mlc gene of the present invention also includes the DNA which codes for a protein having the amino acid sequence including substitution, deletion, insertion, addition or inversion of one to several amino acid residues in the amino acid sequence encoded by the E. coli mlc gene, and has an ability to regulate carbohydrate metabolism. DNA which is hybridizable with the nucleotide sequence of E. coli mlc gene under the stringent conditions or the DNA having homology of 90% or more is preferred, and even more preferably 95% or more, and most preferably 99% or more. The term “stringent conditions” referred to herein as a condition under which so-called specific hybrid is formed, and non-specific hybrid is not formed. For example, the stringent conditions include a condition under which DNAs having high homology, for instance DNAs having homology no less than 70% to each other, are hybridized. Alternatively, the stringent conditions are exemplified by conditions which comprise ordinary condition of washing in Southern hybridization, e.g., 60° C., 1×SSC, 0.1% SDS, preferably 0.1×SSC, 0.1% SDS. Duration of washing procedure depends on the type of membrane used for blotting and, as a rule, is recommended by manufacturer. The time for washing of the Hybond™ N+ nylon membrane (Amersham) in the stringent conditions is preferably between approximately 1 and 15 minutes, more preferably between approximately 5 and 15 minutes, even more preferably between approximately 10 and 15 minutes, and most preferably approximately 15 minutes.

Inactivation of the gene can be performed by conventional methods, such as mutagenesis treatment using UV irradiation or nitrosoguanidine (N-methyl-N′-nitro-N-nitrosoguanidine) treatment, site-directed mutagenesis, gene disruption using homologous recombination or/and insertion-deletion mutagenesis (Datsenko K. A. and Wanner B. L., Proc. Natl. Acad. Sci. USA, 2000, 97:12: 6640-45) which is alternatively called a “Red-driven integration”. Such techniques for gene inactivation are well known in the art.

The L-amino acid producing bacterium of the present invention is not particulary limited and, for example, L-threonine producing bacterium is preferred. Therefore, as a bacterium of the present invention, the L-threonine producing bacterium which is modified to have mlc gene inactivated is particularly preferred.

The bacterium of the present invention may be improved by enhancing the expression of one or more genes involved in the L-threonine biosynthesis. Such genes are exemplified by genes of L-threonine operon, i.e. thr operon, which preferably comprises the mutant thrA gene coding for aspartokinase homoserine dehydrogenase I that is resistant to feed back inhibition by threonine; the thrB gene which codes for homoserine kinase; the thrC gene which codes for threonine synthase. Another preferred embodiment of the bacterium is a bacterium that is modified to have enhanced expression of rhtA gene, which codes for putative transmembrane protein.

Another preferred embodiment is a bacterium that is modified to have enhanced expression of aspC gene, which codes for aspartate aminotransferase (aspartate transaminase) (Russian patent application No. 2002104983). The most preferred embodiment is a bacterium that is modified to have enhanced expression of all of aspC gene, the mutant thrA gene, the thrB gene, the thrC gene and the rhtA gene and modified to inactivate mlc gene.

To achieve enhanced expression of the L-threonine operon, the bacterium can be modified in several ways. For example, the bacterium can be transformed with DNA having genes which are involved in L-threonine biosynthesis, or alternatively, the expression regulation sequence of the bacterium chromosomal DNA can be altered. Even a further method includes, for example, increasing of the gene copy number. Introduction of a gene into a vector that is able to function in a bacterium belonging to the genus Escherichia increases copy number of the gene. Preferably, multi-copy vectors can be used. Examples of multi-copy vector include, but are not limited to, pBR322, pUC19, pBluescript KS+, pACYC177, pACYC184, pAYC32, pMW119, pET22b. The concrete plasmids pVIC40 and pPRT614 (both are derivativies of pBR322) both have genes of the threonine operon and are described in U.S. Pat. Nos. 5,175,107 and 6,132,999, respectively.

A further method of gene expression enhancement includes introduction of multiple copies of the gene into the bacterial chromosome by, for example, homologous recombination, or any other method known to those with skill in the art.

An even further method of gene expression enhancement includes placing the DNA for which enhanced expression is desired under control of a more potent promoter in place of the native promoter. This method can be combined with multiplication of the gene copy number. The strength of a promoter is defined by the frequency of acts of RNA synthesis initiation. Methods for evaluation of promoter strength and examples of potent promoters are described by Deuschle, U., Kammerer, W., Gentz, R., Bujard, H. (Promoters in Escherichia coli: a hierarchy of in vivo strength indicates alternate structures, EMBO J. 1986, 5, 2987-2994). For example the tac promoter is known as a potent constitutive promoter. A strain which has a strong promoter replacing the native promoter is described in U.S. Pat. No. 5,939,307. Other known potent promoters which can be used in the present invention include but are not limited to, are PL promoter, P_{R} promoter, lac promoter, trp promoter, and trc promoter.

Enhancement of translation to increase L-threonine operon activity is also encompassed by the present invention. This can be achieved, for example, by replacing the native Shine-Dalgarno (SD) sequence with a more efficient SD sequence when the SD sequence is a region upstream of the start codon of mRNA interacting with the 16S RNA of ribosome (Shine J. and Dalgarno L., PNAS, USA, 1974, 71(4):1342-6).

As a parent strain of the bacterium of the present invention, the L-threonine producing bacteria belonging to the genus Escherichia such as E. coli strain VKPM B-3996 (U.S. Pat. No. 5,175,107, U.S. Pat. No. 5,705,371), E. coli strain NRRL-21593 (U.S. Pat. No. 5,939,307), E. coli strain FERM BP-3756 (U.S. Pat. No. 5,474,918), E. coli strains FERM BP-3519 and FERM BP-3520 (U.S. Pat. No. 5,376,538), E. coli strain MG442 (Gusyatiner et al., Genetika (in Russian), 14, 947-956 (1978)), E. coli strains VL643 and VL2055 (EP 1149911 A) and the like may be used.

The bacterium of the present invention can be obtained by inactivation of mlc gene in the bacterium inherently having ability to produce L-amino acid. Alternatively, the bacterium of present invention can be obtained by imparting the ability to produce L-amino acid to the bacterium already having mlc gene inactivated.

Methods for preparation of plasmid DNA, digestion and ligation of DNA, transformation, selection of an oligonucleotide as a primer and the like may be ordinary methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

The method of the present invention is a method which produces increased L-amino acid in a culture medium. The method of the present invention comprises the steps of cultivating the bacterium of the present invention in a glucose-containing culture medium to produce and accumulate L-amino acid in the medium, and collecting L-amino acid from the medium. Preferably, the method of the present invention is a method for producing L-threonine, which method comprises the steps of cultivating the bacterium of the present invention in a culture glucose-containing medium to produce and accumulate L-threonine in the medium, and collecting L-threonine from the medium.

In the present invention, the cultivation, the collection and purification of L-amino acid from the medium and the like may be performed in a manner similar to the conventional fermentation method wherein an amino acid is produced using a bacterium.

A medium used for culture may be either a synthetic medium or a natural medium, so long as the medium includes a carbon source and a nitrogen source and minerals and, if necessary, appropriate amounts of nutrients which the bacterium requires for growth. The carbon source may include various carbohydrates such as glucose and sucrose, and various organic acids. A particularly preferred culture medium will contain glucose as the primary carbon source, for example, glucose makes up more than 50%, preferably more than 70%, more preferably more than 90%, of the total carbon source. Depending on the mode of assimilation of the used microorganism, alcohol including ethanol and glycerol may be used. As the nitrogen source, various ammonium salts such as ammonia and ammonium sulfate, other nitrogen compounds such as amines, a natural nitrogen source such as peptone, soybean-hydrolysate, and digested fermentative microorganism may be used.

As minerals, potassium monophosphate, magnesium sulfate, sodium chloride, ferrous sulfate, manganese sulfate, calcium chloride, and the like may be used. As vitamins, thiamine, yeast extract and the like may be used.

The cultivation is performed preferably under aerobic conditions such as a shaking culture, and stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to the accumulation of the target L-amino acid in the liquid medium.

After cultivation, solids such as cells can be removed from the liquid medium by centrifugation or membrane filtration, and then L-amino acid can be collected and purified by ion-exchange, concentration and crystallization methods.

### EXAMPLES

The present invention will be more concretely explained below with reference to following Examples, which are intended to be illustrative only and are not intended to limit the scope of the invention as defined by the appended claims.

### Example 1

### Construction the Strain with Inactivated mlc Gene

Deletion of mlc Gene.

Deletion of mlc gene was performed by means of the method firstly developed by Datsenko and Wanner (Proc. Natl. Acad. Sci. USA, 2000, 97(12), 6640-6645) called as a “Red-driven integration”. According to this procedure, the PCR primers mlcIL (SEQ ID NO: 1) and mlcIR (SEQ ID NO: 2), which are complementary to the region adjacent to the mlc gene or the gene conferring antibiotic resistance to the template plasmid, respectively, were generated. The plasmid pACYC184 (NBL Gene Sciences Ltd., UK) (GenBank/EMBL accession number X06403) was used as a template in PCR reaction. Conditions for PCR were following: denaturation step for 3 min at 95° C.; profile for the first two cycles: 1 min at 95° C., 30 sec at 34° C., 40 sec at 72° C.; profile for the last 30 cycles: 30 sec at 95° C., 30 sec at 50° C., 40 sec at 72° C.; final step: 5 min at 72° C.

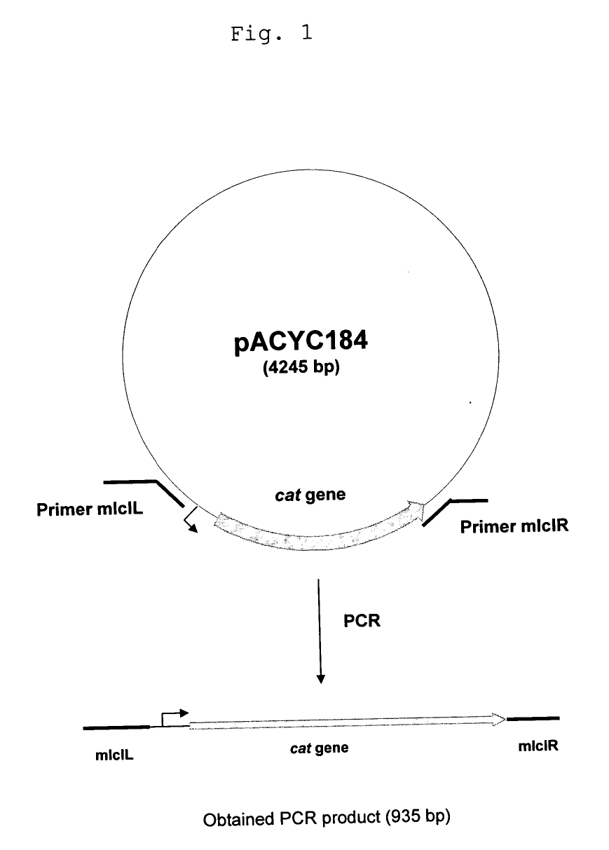

The obtained 935 bp PCR product (FIG. 1, SEQ ID NO: 3) was purified in agarose gel and used for electroporation of the E. coli strain MG1655, containing the plasmid pKD46 with temperature sensitive replication ability. The plasmid pKD46 (Datsenko and Wanner, Proc. Natl. Acad. Sci. USA, 2000, 97:12:6640-45) includes 2,154 nt (31088-33241) DNA fragment of phage X (GenBank accession No. J02459) which contains the genes of λ Red homologous recombination system (γ, β, exo genes) under control of arabinose-inducible P_{araB} promoter. The plasmid pKD46 is necessary for integration of the PCR product into chromosome of the strain MG1655.

Electrocompetent cells were prepared as follows: night culture of E. coli strain MG1655 grown at 30° C. in LB medium supplemented with ampicillin (100 mg/l), was diluted in 100 times with 5 ml of SOB medium (Sambrook et al, “Molecular Cloning A Laboratory Manual, Second Edition”, Cold Spring Harbor Laboratory Press (1989)) containing ampicillin and L-arabinose (1 mM). The obtained culture was grown with aeration at 30° C. to an OD₆₀₀ of ≈0.6 and then made electrocompetent by being concentrated 100-fold and washed three times with ice-cold deionized H₂O. Electroporation was performed using 70 μl of the cells and 100 ng of the PCR product. Cells after electroporation were incubated with 1 ml of SOC medium (Sambrook et al, “Molecular Cloning A Laboratory Manual, Second Edition”, Cold Spring Harbor Laboratory Press (1989)) at 37° C. for 2.5 h and after that plated onto L-agar and grown at 37° C. to select Cm^{R} (chloramphenicol resistant) recombinants. Then to eliminate the pKD46 plasmid, 2 passages on L-agar with Cm (chloramphenicol) at 42° C. were performed and the obtained colonies were tested for sensitivity to ampicillin.

Verification of mlc Gene Deletion by PCR.

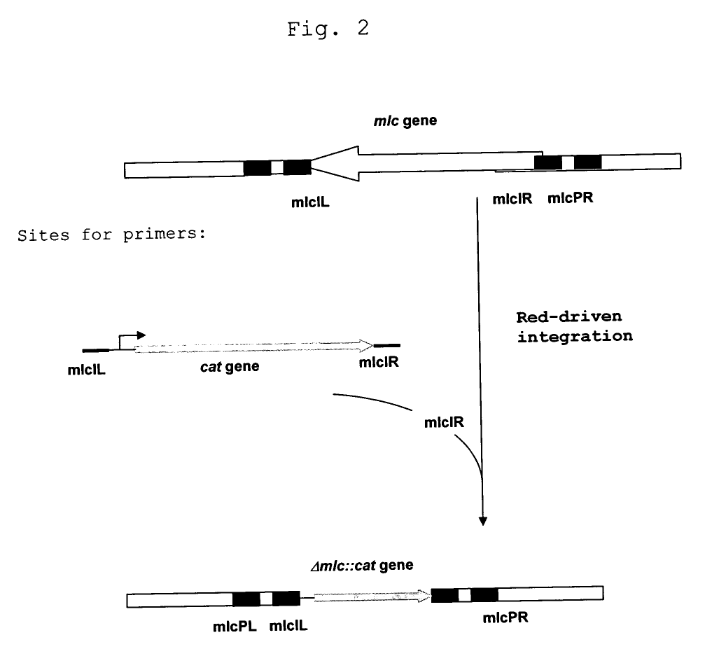

The mutants, containing the deletion of mlc gene, marked with Cm resistance gene, were verified by PCR. Locus-specific primers mlcPL (SEQ ID NO: 4) and mlcPR (SEQ ID NO: 5) were used in PCR for the verification. Conditions for PCR verification were following: denaturation step for 3 min at 94° C.; profile for the 30 cycles: 30 sec at 94° C., 30 sec at 52° C., 2 min at 72° C.; final step: 7 min at 72° C. PCR product, obtained in the reaction with the DNA from the cells of parental Mlc⁺ strain MG1655 as a template, was 1492 nt in length (FIG. 2, SEQ ID NO: 6). PCR product, obtained in the reaction with the DNA from the cells of mutant MG1655 Δmlc::cat strain as a template, was 1191 nt in length (FIG. 2, SEQ ID NO: 7).

Construction of L-threonine Producing Strain With Inactivated mlc Gene.

L-threonine producing strain E. coli TDH7/pRT614 (VKPM B-5318, U.S. Pat. No. 6,132,999) was transduced to Cm resistance by the standard procedure of P1 transduction (Sambrook et al, “Molecular Cloning A Laboratory Manual, Second Edition”, Cold Spring Harbor Laboratory Press (1989)). The strain MG1655 Δmlc::cat was used as a donor. The resulted strain TDH7Δmlc::cat/pRT614 was verified by PCR to have Δmlc::cat deletion by means of primers mlcPL (SEQ ID NO: 4) and mlcPR (SEQ ID NO: 5).

### Example 2

### Production of L-threonine by E. coli Strain With Inactivated mlc Gene

Both E. coli strain TDH7/pRT614 and TDH7Δmlc::cat/pRT614 were grown for 18-24 hours at 37° C. on L-agar plates containing streptomycin (50 μg/ml). Then one loop of the cells was transferred to 50 ml of L-broth of the following composition: tryptone—10 g/l, yeast extract—5 g/l, NaCl—5 g/l. The cells (50 ml, OD₅₄₀-0.12 o.u.) grown at 37° C. for 4 hours on shaker (140 rpm) were used for seeding 450 ml of the medium for fermentation. The batch fermentation was performed in laboratory fermenter having a capacity of 1.01 under aeration (1/1 vvm) with stirring at a speed of 1200 rpm at 39° C. The pH value was maintained automatically at 6.6 using 8% ammonia liquor. The results are presented in Table 1.

The composition of the fermentation medium (g/l):

| | |
|---|---|
| Glucose | 100.0 |
| NH₄Cl | 1.75 |
| KH₂PO₄ | 1.0 |
| MgSO₄.7H₂O | 0.8 |
| FeSO₄.7H₂O | 0.01 |
| MnSO₄.5H₂O | 0.01 |
| Mameno (TN) | 0.15 |
| Betaine | 1.0 |

Glucose and magnesium sulfate are sterilized separately.
pH is adjusted to 6.6.

TABLE 1

| | Amount of | | | |
|---|---|---|---|---|
| | threonine, | | | Cultivation |
| Strain | g/l | Yield, % | DCW, g/l | time, h |
| TDH7/pRT614 | 12.6 | 13.3 | 16.6 | 22.8 |
| TDH7Δmlc::cat/pR | 16.9 | 18.2 | 16.8 | 21.7 |
| T614 | | | | |

As it is seen from Table 1, inactivation of the mlc gene improved the L-threonine accumulation by the L-threonine producing strain TDH7/pRT614.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. An L-amino acid producing bacterium belonging to the genus Escherichia, wherein the bacterium has been modified to have mlc gene inactivated.

2. The L-amino acid producing bacterium according to claim 1, wherein L-amino acid is L-threonine.

3. The L-amino acid producing bacterium according to claim 2, wherein the bacterium has been modified to have enhanced expression of L-threonine operon.

4. A method for producing L-amino acid, which method comprises the steps of:
a) cultivating the bacterium according to of claim 1 in a medium to produce and accumulate L-amino acid in the medium, and
b) collecting L-amino acid from the medium.

5. The method according to claim 4, wherein L-amino acid is L-threonine.

6. An E. coli bacterium comprising an inactive mlc gene, wherein an L-amino acid is produced by said bacterium in a medium containing glucose as the primary carbon source at levels higher than an E. coli bacterium having an active mlc gene.

7. The E. coli bacterium of claim 6 wherein said L-amino acid produced is a member of the aspartate family of amino acids.

8. The E. coli bacterium of claim 7 wherein said L-amino acid produced is L-threonine.

9. The E. coli bacterium of claim 6 wherein said mlc gene has been deleted.

10. The E. coli bacterium of claim 6 wherein said mlc gene has been mutated.

11. The E. coli bacterium of claim 6 wherein the regulatory elements controlling expression of said mlc gene have been mutated.

12. A method of producing an L-amino acid comprising:
a) cultivating an E. coli bacterium comprising an inactive mlc gene in a medium contain glucose as the primary carbon source allowing said L-amino acid to accumulate, and
b) collecting said L-amino acid from the medium,
wherein said E. coli bacterium produces said L-amino acid at levels higher than an E. coli bacterium having an active mlc gene.

13. The method of claim 12 wherein said mlc gene has been deleted.

14. The method of claim 12 wherein said mlc gene has been mutated.

15. The method of claim 12 wherein the regulatory elements controlling expression of said mlc gene have been mutated.

16. The method of claim 12 wherein said L-amino acid produced is a member of the aspartate family of amino acids.

17. The E. coli bacterium of claim 16 wherein said L-amino acid produced is L-threonine.

18. An E. coli bacterium comprising an inactive mlc gene wherein an L-amino acid are produced by said bacterium in a medium containing glucose as the primary carbon source in amounts larger than a wild type E. Coli strain.

19. An E. coli bacterium comprising an inactive mlc gene wherein an L-amino acid is produced by said bacterium in a medium containing glucose as the primary carbon source in amounts larger than a parental E. Coli strain.
